SEQUENCE LISTING (8 sequences)

SEQ ID NO: 1 - DNA, 1212 bp, homo sapiens
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 2 - protein, 403 aa, homo sapiens [sequence omitted]

SEQ ID NO: 3 - DNA, 21 bp, Artificial
  chemically synthesized
aacagtagaggagccgtcaaa

SEQ ID NO: 4 - RNA, 21 nt, Artificial
  chemically synthesized
aacccaccacagcuagaacuu

SEQ ID NO: 5 - RNA, 19 nt, Artificial
  chemically synthesized
aggcacaagaggcccuaga

SEQ ID NO: 6 - RNA, 27 nt, Artificial
  chemically synthesized
guuagcagaaacaaaaggagauaucaa

SEQ ID NO: 7 - DNA, 23 bp, Artificial
  chemically synthesized
cgggaagacaagttcatgtactt

SEQ ID NO: 8 - DNA, 21 bp, Artificial
  chemically synthesized
gatcttgaccaatggctaagt

(19) United States
(12) Patent Application Publication
(10) Pub. No.: US 2007/0297985 A1
(43) Pub. Date: Dec. 27, 2007

(54) METHOD AND COMPOSITION FOR ENHANCING BONE FORMATION

(76) Inventors: Bart O. Williams, Caledonia, MI (US); Thomas L. Clemens, Birmingham, AL (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/733,615

(22) Filed: Apr. 10, 2007

Related U.S. Application Data
(60) Provisional application No. 60/790,881, filed on Apr. 11, 2006.

(51) Int. Cl. A61K 31/70; A61K 31/7105; A61P 19/00; G01N 33/15

(52) U.S. Cl. 424/9.2; 514/43; 514/44

(57) ABSTRACT

A method for screening for an osteogenic compound that is a PTEN antagonist. Also included are methods for enhancing bone formation and pharmaceutical compositions therefore, as well as methods for treating conditions associated with bone loss.

This application claims benefit of provisional application Ser. No. 60/790,881, filed Apr. 11, 2006, entitled A METHOD AND COMPOSITION FOR ENHANCING BONE FORMATION, the entire contents of which are incorporated herein in their entirety.

### GOVERNMENT INTERESTS

The U.S. Government has a paid-up license in this invention and the right in limited circumstances to require the patent owner to license others on reasonable terms as provided for by the terms of (Grant No. NIH IRO1 ARO49410-01A1) awarded by the National Institutes of Health.

### FIELD OF THE INVENTION

This invention in the field of biochemistry and medicine relates to screening for and treatment with bone formation compounds.

### BACKGROUND OF THE INVENTION

The development and maintenance of the mammalian skeleton is controlled by actions of morphogens and growth factors on bone cells. Bone formation is carried out by the osteoblast, a mesenchymal cell whose life span and activity is regulated by growth factor signaling networks (Mundy G R, Boyce B, Hughes D, Wright K, Bonewald L, Dallas S, Harris S, Ghosh-Choudhury N, Chen D, Dunstan C, 1995. The effects of cytokines and growth factors on osteoblastic cells. Bone 17:71S-75S; Karsenty G, 2000. Bone formation and factors affecting this process. Matrix Biol 2000 May;19 (2):85-9 19:85-89). Prominent growth factors regulating osteoblasts are the insulin like growth factors (IGFs), bone morphogenetic proteins (BMPs) and Wnts which can impact osteoblast proliferation and lifespan by activating anti-apoptotic pathways.

Mesenchymal stem cells develop in stages and give rise to osteoblasts, among other cell types. Osteoblast precursors give rise to mature functional osteoblasts. Osteoblasts secrete non-mineralized bone matrix or osteoid, which is composed of approximately ninety percent Type I collagen and ten percent non-collagenous proteins. Osteoblasts deposit osteoid on existing bone surfaces, or defect sites, that mineralize over time forming new bone.

Some evidence suggests that lipid kinase phosphoinositide 3-kinase (PI3K) plays a role in differentiation, survival, and mechanotransduction in osteoblasts. (Golden, L. H., Insogna, K. L., 2004. The expanding role of PI3 kinase in bone, Bone 34:3-12). A decrease and/or delay in osteoblast apoptosis may allow additional sythesis and more bone matrix by osteoblasts, more bone formation and bone mass. (Chaudhary, L. R., Hruska, K. A., Journal of Cellular Biochemistry. 81:304-311). The cell survival signal Akt is differentially activated by PDGF-BB, EGF, and FGF-2 in osteoblastic cells. (Chaudhary, L. R., Hruska, K. A., Journal of Cellular Biochemistry. 81:304-311). Further evidence suggests a role for PI3-K in osteoblast differentiation and survival. (Golden, L. H., Insagna, K. L., 2004. The expanding role of PI3-kinase in bone. Bone 34:3-12). Osteoclasts, in contrast to osteoblasts, are hematopoietic cells responsible for bone resorbtion. (Horowitz, M. C., Bothwell, A. L., Hesslein, G. T., Pflugh, D. L., Schatz, David G., 2005. B cells and osteoblast and osteoclast development. Immunological Reviews, 208:141-153).

PI3K, a critical mediator of multiple signaling pathways, is a key control point in the apoptosis pathway (Cantley L C, 2002. The phosphoinositide 3-kinase pathway 79. Science 296:1655-1657; Cantley L C, Neel B G, 1999. New insights into tumor suppression: PTEN suppresses tumor formation by restraining the phosphoinositide 3-kinase/AKT pathway 135. Proc Natl Acad Sci U S A 96:4240-4245). Receptor tyrosine kinase growth factor receptors become activated and bind to the p85 adaptor/regulatory subunit. This subunit binds to the catalytic subunit (p110) and activates PI3K. PI3K then phosphorylates at the 3 position of the inositol ring of PI4P or PI4,5P2 to generate PI(3, 4)P₂ and PI(3, 4, 5)P₃, respectively. PI(3, 4)P₂ and PI(3, 4, 5)P₃ act as docking sites for intracellular signaling proteins, bring these proteins together in complexes, and relay the signal into the cell. PI(3, 4)P₂ and PI(3, 4, 5)P₃ remain in the plasma membrane until dephosphorylated by inositol phospholipids phosphatases that remove the phosphate from the 3 position of the inositol ring, reversing (negatively regulating) the activity of PI3K. One such inositol phospholipid phosphatase is a dual function lipid and protein phosphatase PTEN (phosphatase and tensin homologue deleted on chromosome 10). PTEN is also known as MMAC1 or TEP1. The human cDNA sequence and amino acid sequence for PTEN are provided herein as SEQ ID NOS.: 1 and 2, respectively. The phosphatase domain of PTEN is provided as amino acids 54-182 of SEQ ID NO.:2. Pten for other species, such as Mus musculus, has been described.

Absent PTEN, a cell has increased levels of PI3K products, increased cell proliferation, and increased resistance to apoptosis. An absence of PTEN has been correlated with Akt activation in cancer cells. Similarly, PTEN mutations in germ line cells result in a hereditary disease known as Cowdens's disease, which carries increased incidence of malignant tumors. (Osake, M., Oshimura, M., and Ito., H., 2004. PI3K-Akt pathway: its functions and alterations in human cancer. Apoptosis, 9:667-676.)

Important downstream targets of PI3K and of PIP3 include the serine-threonine Akt kinase family (also known as PKB). PIP3 generated in the plasma membrane recruits Akt and PDK1 to the plasma membrane through an interaction between the phosphoinositide and the Akt or PDK1 pleckstrin homology domains (PH). Once recruited to the plasma membrane, Akt is phosphorylated and activated by PDK1.

Akt promotes both cell growth and cell survival by inactivating its downstream substrates including GSK3, BAD, FOXO and TSC2 (Downward J, 2004. PI 3-kinase, Akt and cell survival. Semin Cell Dev Biol 15:177-182). Studies in Caenorhabditis elegans and Drosophilla melanogaster have linked activation of Akt to regulation of certain FOXO transcription factors and to the activation of mTOR and p70S6K. By contrast, loss of PTEN function either in embryonic stem cells or in human cancer cell lines results in accumulation of PtdIns(3,4,5)P3 and activation of its downstream signaling molecule, Akt/PKB. Activation of the PI 3-kinase/Akt pathway in turn stimulates cell cycle progression, cell survival and cell migration (Liliental J, Moon S Y, Lesche R, Mamillapalli R, Li D, Zheng Y, Sun H, Wu H. I., Genetic deletion of the Pten tumor suppressor gene promotes cell motility by activation of Rac1 and Cdc42 GTPases Curr Biol. 2000 Apr 6;10(7):401-4.; Stambolic V, Suzuki A, de la Pompa J L, Brothers G M, Mirtsos C, Sasaki T, Ruland J, Penninger J M, Siderovski D P, Mak T W. Negative regulation of PKB/Akt-dependent cell survival by the tumor suppressor PTEN, Cell. 1998 Oct 2;95(1):29-39. +; Sun H, Lesche R, Li D M, Liliental J, Zhang H, Gao J, Gavrilova N, Mueller B, Liu X, Wu H., PTEN modulates cell cycle progression and cell survival by regulating phosphatidylinositol 3,4,5,-trisphosphate and Akt/protein kinase B signaling pathway, Proc Natl Acad Sci U S A. 1999 May 25;96(11):6199-204 One function of activated Akt is inhibition of apoptosis.

There are many conditions and diseases associated with bone loss, such as osteoporosis (a disease manifesting as low bone density). Other conditions and diseases that involve bone loss include juvenile osteoporosis, osteogenesis imperfecta, hypercalcemia, hyperparathyroidism, osteomalacia, osteohalisteresis, osteolytic bone disease, osteonecrosis, Paget's bone disease, and osteoporosis pseudoglioma. Bone loss also is the result of various other diseases or treatments for diseases such as bone loss due to chemotherapy or corticosteroid treatment, as well as bone loss due to rheumatoid arthritis or inflammatory arthritis, osteomyelitis, periodontal bone loss, age-related bone loss, and other forms of osteopenia.

Current strategies to treat bone loss are aimed at inhibiting functions of osteoclasts, the cells that degrade bone. Many such treatments, however, have significant side effects or are not recommended for long-term use. One agent for inhibiting osteoclasts is a (bis) phosphonate that incorporates preferentially into bone because of the calcium phosphonate. Another agent is a compound that mimics the actions of parathyroid hormone. Such compound, however, carries a warning label due to results suggesting it can induce osteosarcoma. As described below, the inventors have developed screening methods, pharmaceutical compositions, and treatments to enhance bone formations by antagonizing PTEN function.

### SUMMARY OF THE INVENTION

The present invention includes a method of screening osteogenic compounds including the following steps: (1) selecting a compound that may be a PTEN antagonist; (2) performing a bone formation assay for the compound; and (3) concluding from the assay whether the compound is osteogenic. In one embodiment of this method, the selected compound antagonizes PTEN in an osteoblast or osteoblast precursor. The bone formation assay may measure bone mass, bone volume, osteoblast number, or osteoblast survival. In this screening method, the conclusion from the bone formation assay is that the selected compound is osteogenic. In another embodiment, the selected compound inactivates PTEN, is an siNA for Pten, is an siNA specific to the phosphatase domain of PTEN, or is an shRNA.

The present invention further includes a pharmaceutical composition for enhancing bone formation utilizing a compound to lower the phosphatase activity of PTEN in osteoblasts or osteoblast precursors. In one embodiment of the composition, the compound inactivates PTEN in osteoblasts or osteoblast precursors. In another embodiment the compound is an siNA for PTEN. In further compositions of the present invention, the siNA is specific to the phosphatase domain of PTEN, or the siNA is an shRNA.

The present invention further includes a method of enhancing bone formation in a mammal comprising administering to the mammal an effective amount of an osteoblast-specific PTEN antagonist. In another embodiment of this method, the osteoblast-specific PTEN antagonist inactivates PTEN in osteoblasts or osteoblast precursors in the mammal. In further methods of the present invention, the osteoblast-specific PTEN antagonist is an siNA for PTEN, the siNA is specific to the phosphatase domain of PTEN, or the siNA is an shRNA.

The present invention also includes a method of treating a condition associated with loss of bone mass comprising administering a pharmaceutical compound that reduces the phosphatase activity of PTEN in osteoblasts or osteoblast precursors. In another embodiment, the compound is administered in an amount effective to enhance bone formation, such as increasing bone mass, bone volume, osteoblast number, or osteoblast survival. Further, in the present method, the compound is an siNA for PTEN that may be specific to the phosphatase domain of PTEN, and may be an shRNA.

### BRIEF DESCRIPTION OF THE DRAWINGS

FIGS. 1A and 1B depict the strategy for conditional disruption of Pten. FIG. 1A is a diagram of the human osteocalcin (hOC) construct used to generate mouse lines expressing a Cre transgene. The arrow indicates the transcriptional orientation. FIG. 1B is a diagram of the generation of a loxP-flanked Pten allele as follows: a) Genomic structure of the mouse Pten gene; b) The targeting vector. Exons 4 and 5 were flanked by two loxP sequences, shown as black arrowheads. A third loxP sequence was introduced to flank the Hygr gene (hygromycin); and c) The mutated allele containing three loxP sequences and the Hygr gene. Cre-mediated deletion was expected to produce the d) Ptenflox allele and e) PtenΔ allele. ( A, Apal; E, EcoRI; H, Hind III; [symbol] ; loxP; Hyg, hygromycin). FIG. 1C shows allele-specific PCR detection of the ΔPten allele in various tissues of mutant and control mice.

FIGS. 2A and 2B are bar graphs showing progressive increase in bone mineral density in male and female mice (respectively) carrying an osteoblast-specific deletion of the Pten gene as compared to control mice.

FIGS. 3A-D are images of microCT analysis performed on samples obtained from twelve month old ΔPten and control mice at the femur (sagital view) in FIG. 3A; midshaft (cross-sectional view) in FIG. 3B; distal femur (cross-sectional view) in FIG. 3C; and calvaria (cross-sectional view) in FIG. 3D. FIGS. 3E-H are graphs showing bone volume/tumor volume, trabecular thickness, trabecular separation, and trabecular number, respectively, of ΔPten and control mice at twelve months of age.

FIGS. 4A and B show that loss of Pten in osteoblasts is associated with increased calvaria thickness and serum alkaline phosphatase (ALP) levels. FIG. 4A are micro CT images performed on samples obtained from the calvaria of 12-month-old control, Pten^{+/-} (heterozygous for ΔPten in osteoblasts), and ΔPten mice. FIG. 4B is a graph showing ALP levels (units/liter) in control, Pten^{+/-}, and ΔPten mice. *P<0.05 SEM is represented by error bars.

FIGS. 5A-E show disruption of the Pten gene increases bone formation rate. FIG. 5A shows fluorescence images from seven-week-old male ΔPten and control mice labeled with sequential doses of calcein. FIGS. 5B-E are bar graphs depicting bone formation rate/bone volume, mineral apposition rate, osteoblast surface/bone surface, osteoclast number/bone perimeter, respectively, of control mice (left bars) and ΔPten mice (right bars).

FIGS. 6A and 6B show disruption of Pten in osteoblasts in vitro activates the Akt pathway. FIG. 6A is a bar graph showing real-time PCR measurements of relative Pten gene expression in primary osteoblast cell cultures from Pten^{flox/flox} calvaria 48 hours after infection. FIG. 6B includes Western blots showing that disruption of PTEN in vitro constitutively activates the Akt pathway.

FIGS. 7A-F show loss of Pten increases osteoblast proliferation and reduces apoptosis. FIG. 7A is a density plot showing loss of Pten increases BrdU incorporation. FIG. 7B is a histogram showing Pten deletion reduces osteoblast apoptosis. FIG. 7C is a bar graph showing the percentage of BrdU incorporated cells in PTEN deletion (with AdCreM1) and Control (with AdGFP) osteoblasts (n=3, p-value=0.000315). FIG. 7D is a bar graph showing the percentage of Annexin V positive cells in Pten deletion (with AdCreM1) and Control (with AdGFP) osteoblasts (n=3, p-value=1.21E-5). FIG. 7E is a graph showing Pten deletion increases forward scatter (FSC) of osteoblast cells. Pten deficient cells are represented by the trace shifted to the right. FIG. 7F is a bar graph showing the mean FSC of control and ΔPten osteoblasts. * means significantly different from control at P<0.05.

FIGS. 8A through 8C show loss of Pten accelerates osteoblast differentiation in vitro. FIGS. 8A and 8C are bar graphs showing the effect of Pten deletion on gene expression at 48 hours (FIG. 8A) and 21 days (FIG. 8C). FIG. 8B shows the effect of Pten deletion on monolayers of osteoblasts stained for alkaline phosphatase expression at 10 days (left panel) and 21 days (right panel) of culture.

### DETAILED DESCRIPTION OF THE PREFERRED EMBODIMENTS

The preferred embodiments of the present invention may be understood more readily by reference to the following detailed description of the specific embodiments and the Examples and Sequence Listing included hereafter.

The Sequence Listing filed concurrently with this application (as a text file named “VAN67P310A.txt” was created on Apr. 10, 2007, has a file size of 8,192 kilobytes, with machine format IBM-PC, operating system capability of MS-Windows, is incorporated herein by reference.

As used herein, the term “an effective amount” means an amount of the compound or pharmaceutical composition that produces a statistically significant result. For example, with respect to the present invention, an effective amount of a compound or pharmaceutical composition means an amount that provides a statistically significant increase in bone formation; or the stabilization of or reversal of a disease or condition associated with a loss of bone mass. An effective amount can be determined using known techniques, depending upon variables such as the particular disease or condition treated, the patient, the patient condition, the method of administration, the formulation, and other factors. An effective amount is demonstrated by a statistically significant difference in bone formation between a treatment group and a control group.

As used herein, the term “treatment” means prophylaxis and therapy. Thus, methods of treatment of the present invention include treatments to prevent or inhibit bone loss, or to stabilize or reverse conditions or diseases associated with bone loss. Treatment also includes therapy for major and minor bone fractures.

All references, patents, patent publications, articles, and databases, referred to in this application are incorporated-by-reference in their entirety, as if each were specifically and individually incorporated herein by reference.

Bone formation is carried out by the osteoblast, a mesenchymal cell whose life span and activity is regulated by growth factor signaling networks. Growth factors activate phosphatidylinositol 3-kinase (PI3K) which enhances cell survival and antagonizes apoptosis via activation of Akt/PKB. This process is negatively regulated by the PTEN phosphatase, which inhibits the activity of PI3K. The inventors investigated the effects of Akt activation in bone in vivo by conditionally disrupting the Pten gene in osteoblasts using Cre mediated recombination. Specifically, to directly investigate the role of Pten in osteoblasts in vivo, the inventors disrupted the gene encoding Pten using Cre mediated deletion techniques.

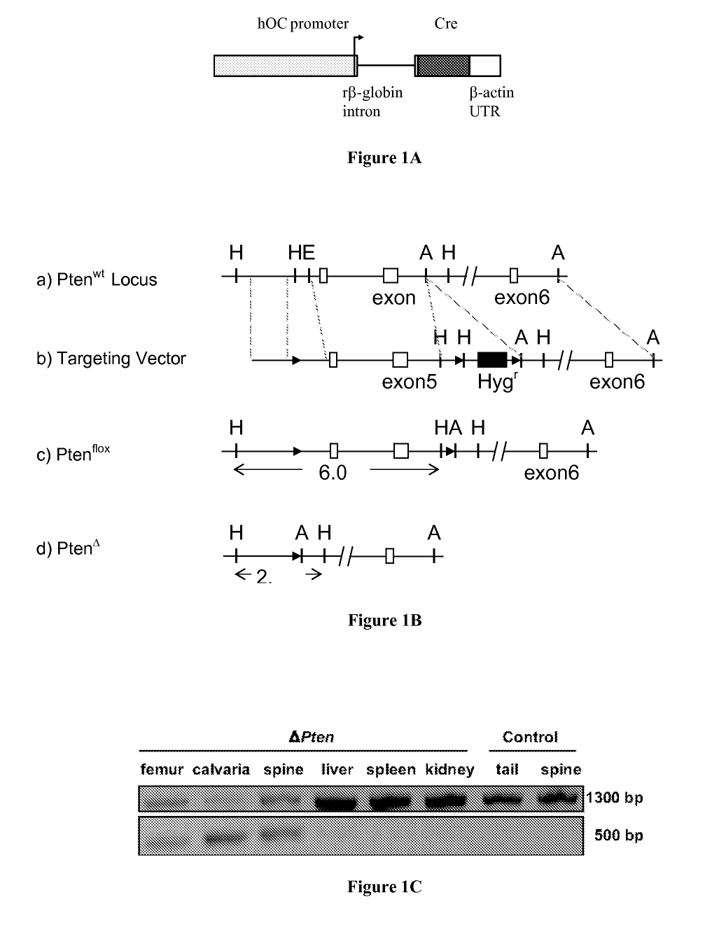

Among the progeny of crosses between OC-cre/Pten^{+/flox} and Pten^{flox/flox} mice, female animals with an OC-cre/Pten^{flox/flox} genotype were selected for a detailed study (referred to hereafter as ΔPten mice), although in all cells other than osteoblasts the floxed alleles of the Pten gene remained intact. PCR analysis by using DNA templates from tissues of ΔPten offspring confirmed that Cre-mediated recombination occurred exclusively in bone (FIG. 1C). The upper panel of FIG. 1C shows PCR fragments generated for the Pten-floxed allele. The lower panel of FIG. 1C shows Pten deletion (ΔPten) allele. Deletion of exons 4 and 5 was detected only in skeletal tissues. Also, by convention, the genetically modified, but normal (control) mice were considered as indistinguishable from wild-type. All mice carrying the bone specific mutation were born alive with the expected Mendelian genotypic frequency.

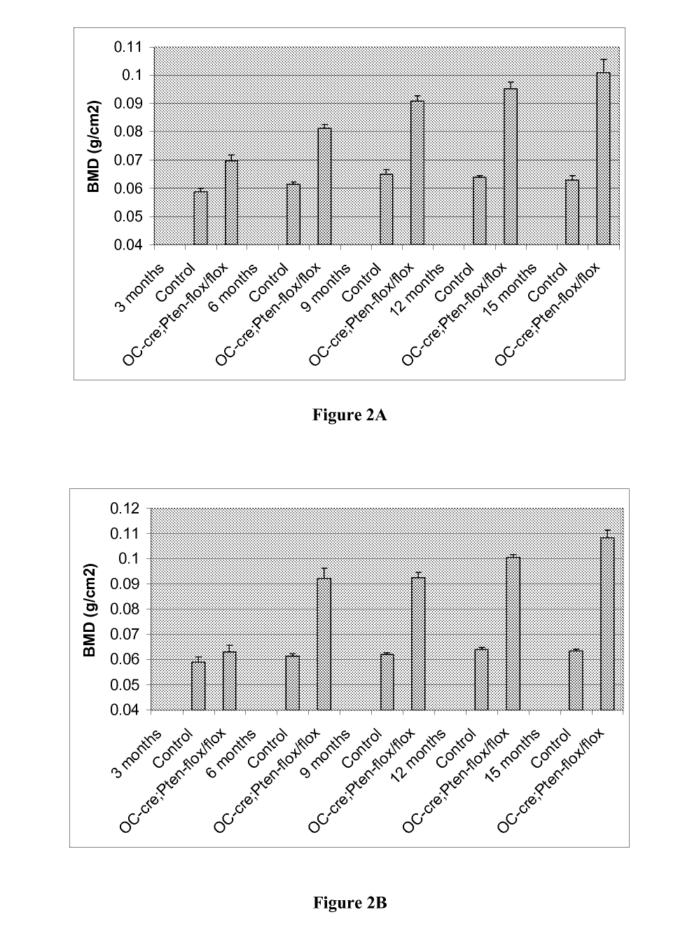

To assess bone mass, the inventors generated cohorts of ΔPten and control mice and performed a longitudinal analysis of their bone mineral density (BMD) via the use of dual energy X-ray absortiometry (DEXA). Measurements were taken at 3, 6, 9, 12, and 15 months of age (FIGS. 2A and 2B). At 3 months of age, both males (FIG. 2A) and females (FIG. 2B) displayed significant increases in BMD relative to their wild type cohorts (p=0.002 in both cases); female mice had a 7% increase in BMD while males displayed a 19% increase. This increase in BMD became progressively greater as the animals aged. By 15 months of age, female OC-cre/Pten^{flox/flox} mice had 71% higher BMD than controls (p=2×10⁻⁶) while BMD in males was increased by 60% (p=5×10⁻⁵). The error bars represent the values of the standard errors of the mean (FIGS. 2A and 2B). At least five mice in each category were examined. The inventors also found that six week old OC-cre/Pten^{flox/flox} mice showed increased BMD (p=0.002) (data not shown).

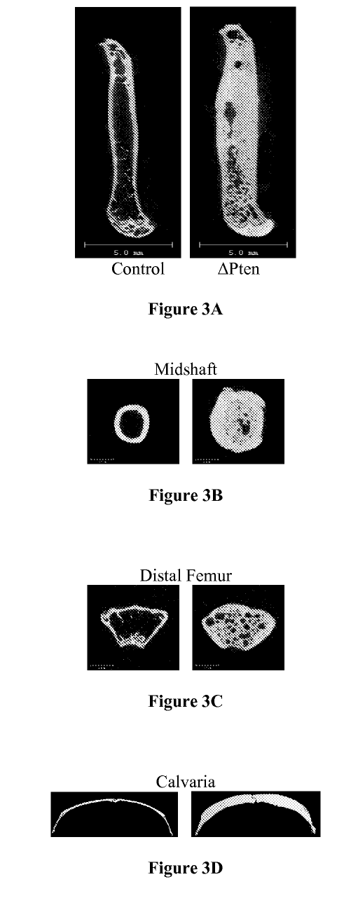

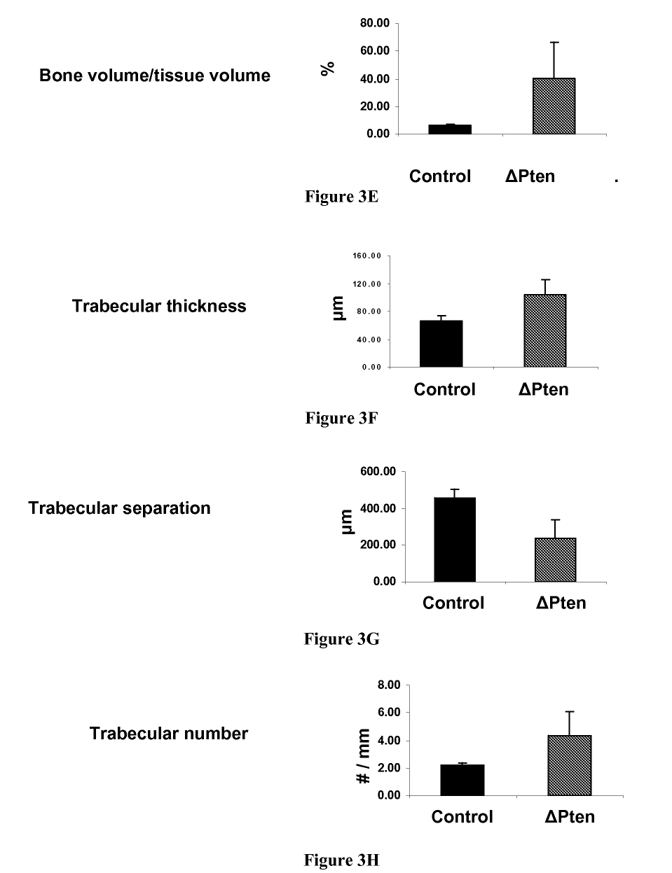

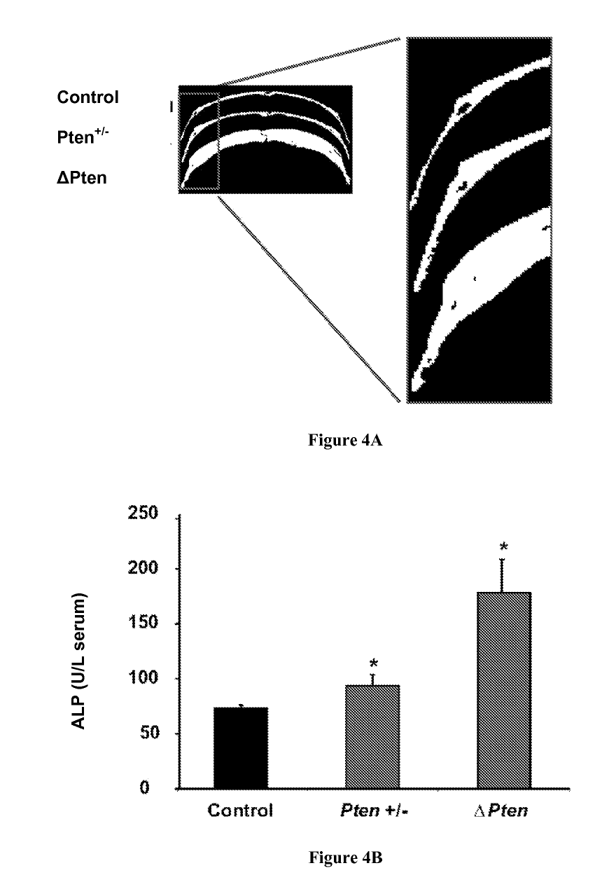

To examine the effect of the loss of Pten on bone architecture, micro CT measurements were performed on female ΔPten mice and control littermates at 3 and 12 months of age. Pten mutant mice demonstrated a striking increase in bone volume at all skeletal sites (FIG. 3A-D). Error bars represent standard error of the mean. This was reflected by a decrease in trabecular separation and an increase in the number of trabeculae (FIGS. 3G and 3H, respectively). In addition, in ΔPten mice, trabecular connectivity was greater than normal. Cortical bone volume and cortical thickness measured at the mid-shaft of the femur were also significantly increased in the mutant mice. The thickness of calvarial bone was similarly increased in the mutants suggesting that loss of Pten influences the development of bone formed through intramembranous processes. (FIG. 4A). Calvaria from mice heterozygous for the conditional Pten allele (OC-Cre^{TG/+}; Pten^{flox/+}) were slightly but consistently thicker than those of controls (FIG. 4A). In addition, serum alkaline phosphatase levels in the heterozygous mice were intermediate between the wild-type and homozygous mutant animals (FIG. 4B). These results indicate that loss of a single Pten allele results in increased bone mass.

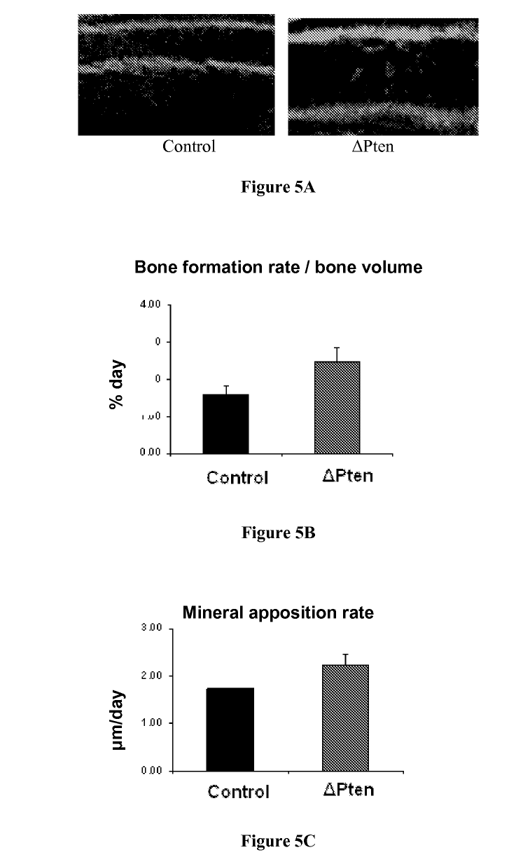

To assess the impact of the Pten mutation on bone formation rates, dynamic histomorphometric measurements on 6 week-old mice doubly labeled with sequential doses of calcein. FIG. 5A depicts the increased bone formation rate for ΔPten mice as compared to the control mice. Cancellous bone formation rate and mineral apposition rate was significantly increased in the ΔPten mice compared to controls (FIGS. 5B and 5C, respectively). In addition, the number of osteoblasts lining bone surfaces was increased (FIG. 5D).

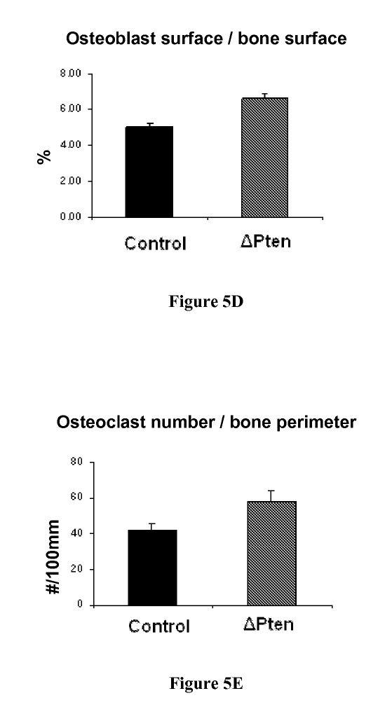

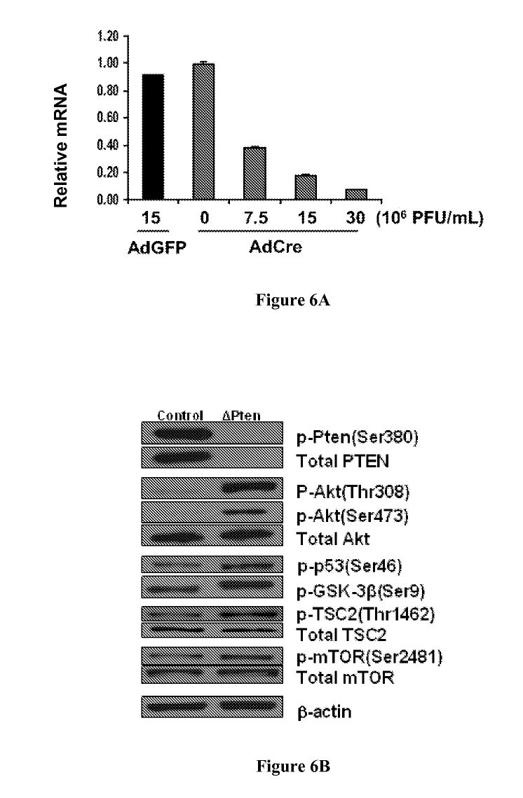

Important downstream targets of PI3K and of PIP3 include the serine-threonine Akt kinase family (also known as PKB). Activation of Akt promotes both cell growth and cell survival by regulation of certain FOXO transcription factors and the activation of mTOR and p70^{S6K}. To examine the effect of Pten loss on signaling pathways downstream of Akt, the inventors analyzed the expression of several key Akt targets following Cre-mediated disruption of the Pten gene in calvarial osteoblasts in vitro. Cells derived from mice carrying the floxed alleles were infected with adenovirus expressing the Cre recombinase (Cre+) or a control adenovirus directing the expression of green fluorescent protein (Cre−) and then cultured in the presence of -glycerol phosphate and ascorbate (mineralizing medium). Infection with 20 MOI was sufficient to decrease Pten mRNA to 10% that observed in GFP infected cells. (FIG. 6A). Primary Pten^{flox/flox} osteoblasts were infected with the indicated number of plaque-forming units of Adeno-Cre or Adeno-GFP virus. Pten normally inactivates PI3K a critical mediator of multiple signaling pathways. To examine the effect of Pten deficiency on these downstream targets, primary calvarial osteoblast expressing or deficient in Pten were deprived of serum for 24 hours and then subjected to immunoblotting to assess expression of Akt and other signaling targets downstream of the PI3 kinase pathway. Whole-cell lysates were extracted from Pten deleted osteoblasts (with AdCreM1, an adenovirus carrying Cre recombinase) or control cells (with AdGFP, a control adenovirus) and analyzed for levels of Pten, phosphorylated Akt, total Akt, phosphorylated GSK-3β, phosphorylated TSC2, total TSC2, phosphorylated mTOR, and total mTOR.

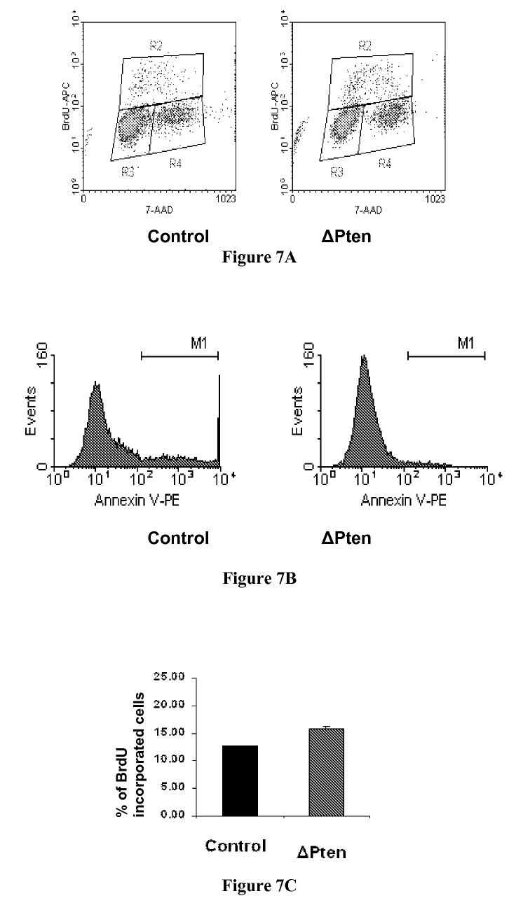

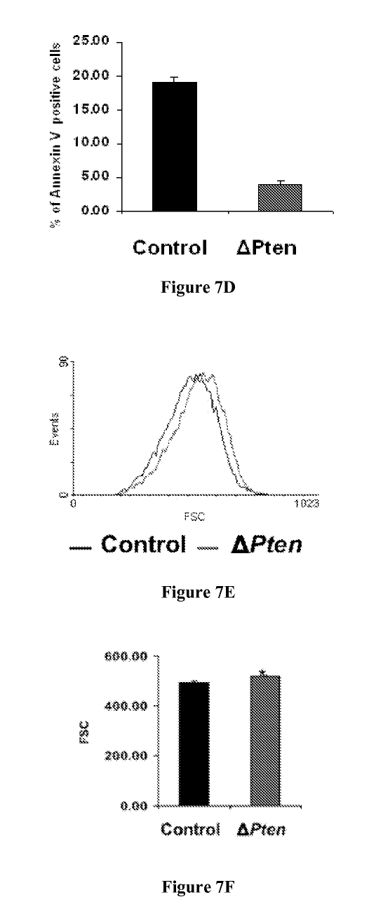

As shown in FIG. 6B, osteoblast deficient in Pten had greatly increased phospho Akt, and mTOR. Increased phosphorylation of the indicated amino acids within Akt (Thr308 and Ser473) is associated with activation of Akt kinase activity (Hennessy B T, Smith D L, Ram P T, Lu Y, and Mills G B, 2005. Exploiting the PI3K/AKT pathway for cancer drug discovery, Nature Reviews Drug Discovery, 4:988-1004.). Consistent with this, dramatic elevations of the levels of the phosphorylation of these resides are present in ΔPten osteoblasts relative to controls. The total levels of Akt are similar in both the ΔPten and control osteoblasts. Several markers of pathways activated downstream of Akt signaling are also shown including increased phosphorylation of p53, GSK3β, TSC2, and mTOR (Hennessy B T, Smith D L, Ram P T, Lu Y, and Mills G B, 2005. Exploiting the PI3K/AKT pathway for cancer drug discovery, Nature Reviews Drug Discovery, 4:988-1004). Further, osteoblasts deficient in Pten proliferated slightly faster that controls when grown in 10% serum (FIGS. 7A and 7C) as measured by BrdU incorporation. Primary osteoblasts from mice carrying floxed Pten alleles were infected with adeno CreM1 virus (MOI 100, 48 hours). Osteoblasts were cultured in alpha-MEM with 1% serum for 24 hours and alpha-MEM with 10% serum for 12 hours before harvest cells for BrdU and 7-AAD staining. FIG. 7A shows cell population of G0/G1 (Region R3), G2/M ( Region R4) and S phase (Region R2). In addition, the rate of apoptosis as measured by annexin staining was markedly reduced in the mutant osteoblasts as compared to the cells from wild type mice (FIGS. 7B and 7D). Osteoblasts infected with either adeno CreM1 or adeno GFP were cultured in alpha-MEM with 1% serum for 24 hours and serum-free alpha-MEM for 12 hours. Cells were then harvested and stained for Annexin V and and 7-AAD and analyzed by FACACalibur. FIG. 7B shows Annexin V positive cells (M1). These results are compatible with the finding of increased osteoblast numbers in the Pten mutant mice and suggest that deficiency in Pten increases osteoblast proliferation and reduces apoptosis. Pten-deficient osteoblasts also exhibited a significant increase in cell size (FIG. 7E and F), consistent with the effects of activation of the Akt and mTOR pathways (Backman, S, Stambolic, V & Mak, T. (2002) Curr Opin Neurobiol 12, 516-522; Plas, D R & Thompson, C B. (2005) Oncogene 24, 7435-7442).

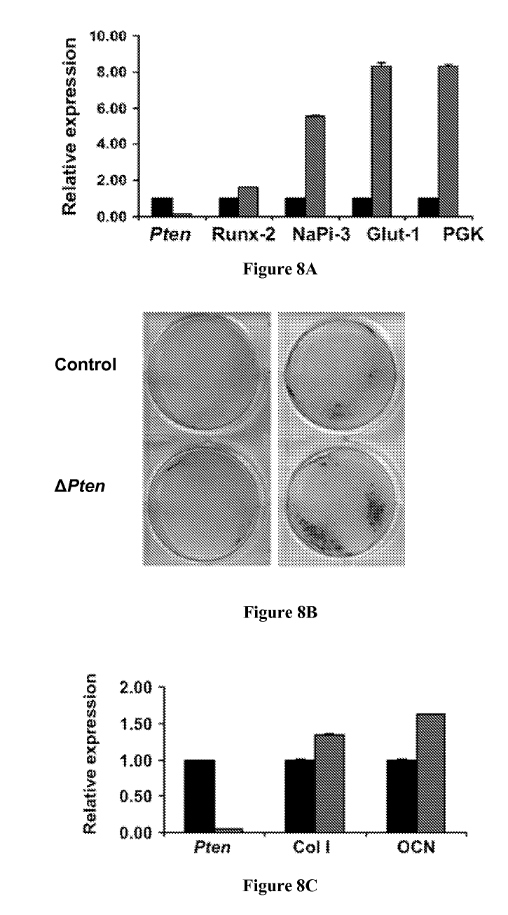

To determine the effect of loss of Pten on osteoblast differentiation, cells derived from mice carrying the floxed alleles were infected with adenovirus expressing the Cre recombinase (Cre+) or a control adenovirus directing the expression of GFP (Cre−), and then differentiated in the presence of β-glycerol phosphate and ascorbate (mineralizing media). Osteoblasts deficient in Pten showed increased expression of several genes, including Runx-2, NaPi3, Glut-1, and PGK, which are expressed early in the differentiation of primary mouse osteoblasts (FIG. 8A). At later times, disruption of Pten was associated with increased alkaline phosphatase expression (FIG. 8B) and augmented expression of gene markers for the differentiated osteoblast, including collagen I and osteocalcin (FIG. 8C). Overall, these results are compatible with the increased numbers and activity of osteoblasts seen in mice lacking Pten in vivo. Calcium deposition was measured by alkaline phosphatase (AP) staining and mineralized nodule formation was analyzed by von Kossa staining in Pten deletion (with AdCreM1, MOI 100) or control cells (with AdGFP, MOI 100) for 48 hours or 21 days culture (FIGS. 8A and 8C, respectively), in the presence of ascorbic acid and β-glycerol phosphate. Relative expression of the indicated mRNAs in osteoblasts expressing (black bars) or lacking (gray bars) Pten was determined by real-time PCR. Col I, collagen I; OCN, osteocalcin.

In accordance with the present invention, the inventors have discovered that compounds antagonizing PTEN can be administered in an amount and manner such that they enhance bone formation. As used herein, “enhanced bone formation” means any of the following: an increase in bone mass, bone volume, osteoblast number, or osteoblast survival. The present invention also includes methods for treating diseases or conditions associated with bone loss. An example of a pharmaceutical composition of the present invention is an siNA for PTEN. As used herein, the term siNA (small, or short, interfering nucleic acid) is meant to be equivalent to other terms used to describe nucleic acid molecules that are capable of mediating sequence specific RNAi (RNA interference), for example short (or small) interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), translational silencing, and others. RNAi is a well known tool for knocking out gene function in a mammal. Preferably, the siNA would include an internal promoter driving the expression of the siNA only in osteoblasts. For example, an osteocalcin promoter could be used for this purpose. Osteocalcin is the product of the last gene that is expressed in an osteoblast, and is responsible for calcifying the bone matrix.

siRNAs suppress gene expression through a highly regulated enzyme-mediated process called RNA interference (RNAi) (Sharp, P. A., Genes Dev. 15:485-490 (2001); Bernstein, E et al., Nature 409:363-366 (2001); Nykanen, A et al., Cell 107:309-321 (2001); Elbashir, S. M. et al., Genes Dev. 15:188-200 (2001)). RNAi involves multiple RNA-protein interactions characterized by four major steps: assembly of siRNA with the RNA-induced silencing complex (RISC), activation of the RISC, target recognition and target cleavage. These interactions may bias strand selection during siRNA-RISC assembly and activation, and contribute to the overall efficiency of RNAi (Khvorova, A et al., Cell 115:209-216 (2003); Schwarz, D S et al. 115:199-208 (2003))).

Two publications that describe preferred approaches and algorithms for selecting siRNA sequences are: Far, R K et al., Nuc Acids Res, 2003, 314417-4424 and Reynolds, A et al., Nature Biotech. 2004, 22:326-330. Far et al. suggests options for assessing target accessibility for siRNA and supports the design of active siRNA constructs. This approach could be automated, adapted to high throughput and is open to include additional parameters relevant to the biological activity of siRNA. To identify siRNA-specific features likely to contribute to efficient processing at each of the steps pf RNAi noted above, Reynolds et al., supra performed a systematic analysis of 180 siRNAs targeting the mRNA of two genes. Eight characteristics associated with siRNA functionality were identified: low G/C content, a bias towards low internal stability at the sense strand 3′-terminus, lack of inverted repeats, and sense strand base preferences (positions 3, 10, 13 and 19). Application of an algorithm incorporating all eight criteria significantly improves potent siRNA selection. This highlights the utility of rational design for selecting potent siRNAs that facilitate functional gene knockdown.

Knockdown of PTEN by various PTEN siNAs (SEQ ID NOS: 3-8) has been confirmed in vitro by others. Kawaguchi, T; et al., 2006. The PTEN/Akt pathway Dictates the Direct αᵥβ₃-Dependent Growth-Inhibitory Action of an Active Fragment of Tumstatin in Glioma Cells in vitro and in vivo. Cancer Res 66(23) 11331-11340; Nho, R; et al., 2006. PTEN Regulates Fibroblast Elimination during Collagen Matrix Contraction. J Biol Chem 281(44) 33291-33301; van Duijn, P; et al., 2006. PI3K/Akt Signaling Regulatesp27^{kip1} Expression Via Skp2 in PC3 and DU145 Prostate Cancer Cells, but is not a Major Factor in p27^{kip1}Regulation in LNCaP and PC346Cells. Prostate 66(7) 749-760; Edwin, F; et al., 2006. The Tumor Suppressor PTEN Is Necessary for Human Sprouty 2-mediated Inhibition of Cell Proliferation. J Biol Chem 281(8) 4816-4822; Hjelmeland, A; et al., (2005). Transforming Growth Factor B-Mediated Invasion with Enhanced SMAD3 Transcriptional Activity. Cancer Res 65(24) 11276-11281. Other candidate siRNA sequences against human PTEN could be selected using a process that involves running a BLAST search against the sequence of PTEN, and selecting sequences that survive to ensure that these sequences will not be cross matched with any other genes.

siRNA sequences could be cloned into an expression plasmid or vector and tested for their activity in abrogating PTEN function in PTEN-expressing cells. Such reduced PTEN activity could be assessed using the assays described herein to test for: increased phospho-Akt; increased phosphorylation of p53, GSK3β, TSC2, and mTor; increased osteoblast proliferation and cell size; increased expression of Runx-2, NaPi3, Glut-1, and PGK.

In a most preferred embodiment, the inhibitory molecule is a double stranded nucleic acid (preferably an RNA), used in a method of RNA interference. RNA interference is the sequence-specific degradation of homologues in an mRNA of a targeting sequence in an siNA. Long double stranded interfering RNAs, such a miRNAs, appear to tolerate mismatches more readily than do short double stranded RNAs. In addition, as used herein, the term RNAi is meant to be equivalent to other terms used to describe sequence specific RNA interference, such as post transcriptional gene silencing, or an epigenetic phenomenon. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure and thereby alter gene expression (see, for example, Allshire (2002) Science 297, 1818-1819; Volpe et al. (2002) Science 297, 1833-1837; Jenuwein (2002) Science 297, 2215-2218; and Hall et al. (2002) Science 297, 2232-2237.)

An siNA can be designed to target any region of the coding or non-coding sequence of an mRNA. An siNA is a double-stranded polynucleotide molecule comprising self-complementary sense and antisense regions, wherein the antisense region comprises nucleotide sequence that is complementary to nucleotide sequence in a target nucleic acid molecule or a portion thereof and the sense region has a nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary. The siNA can be assembled from a single oligonucleotide, where the self-complementary sense and antisense regions of the siNA are covalently linked by means of a nucleic acid based or non-nucleic acid-based linker(s) as is known in the art, or are alternately non-covalently linked by ionic interactions, hydrogen bonding, Van der Waal's interactions, hydrophobic interactions, and/or stacking interactions. The siNA can be a polynucleotide with a hairpin secondary structure, having self-complementary sense and antisense regions. The siNA can be a circular single-stranded polynucleotide having two or more loop structures and a stem comprising self-complementary sense and antisense regions, wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (or can be an siNA molecule that does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al. (2002) Cell 110, 563-574 and Schwarz et al. (2002) Molecular Cell 10, 537-568), or 5′,3′-diphosphate.

As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. In certain embodiments, short interfering nucleic acids do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” Other chemical modifications, e.g., as described in PCT/US03/05346 and PCT/US03/05028, can be applied to any siNA sequence of the invention.

Preferably a molecule mediating RNAi has a 2 nucleotide 3′ overhang. If the RNAi molecule is expressed in a cell from a construct, for example from a hairpin molecule or from an inverted repeat of the desired sequence, then the endogenous cellular machinery will create the overhangs.

Considerations to be taken into account when designing an RNAi molecule include, e.g., the sequence to be targeted, secondary structure of the RNA target and binding of RNA binding proteins. Methods of optimizing siRNA sequences will be evident to the skilled worker. Typical algorithms and methods are described in Vickers et al. (2003) J Biol Chem 278:7108-7118; Yang et al. (2003) Proc Natl Acad Sci USA 99:9942-9947; Far et al. (2003) Nuc. Acids Res. 31:4417-4424; and Reynolds et al. (2004) Nature Biotechnology 22:326-330.

Methods of making siRNAs are conventional. In vitro methods include processing the polyribonucleotide sequence in a cell-free system (e.g., digesting long dsRNAs with RNAse III or Dicer), transcribing recombinant double stranded DNA in vitro, and, preferably, chemical synthesis of nucleotide sequences homologous to the Pten sequence. See, e.g., Tuschl et al. (1999) Genes & Dev. 13:3191-3197.

In vivo methods include
(1) transfecting DNA vectors into a cell such that a substrate is converted into siRNA in vivo. See, for example, Kawasaki et al. (2003) Nucleic Acids Res 31:700-707; Miyagishi et al. (2003) Nature Biotechnol 20:497-500; Lee et al. (2002) Nature Biotechnol 20:500-505:Brummelkamp et al. (2002) Science 296:550-553; McManus et al. (2002) RNA 8:842-850; Paddison et al. (2002) Gene Dev 16:948-958; Paddison et al. (2002) Proc Natl Acad Sci USA 99:1443-1448); Paul et al. (2002) Nature Biotechnol 20:505-508; Sui et al. (2002) Proc Natl Acad Sci USA 99:5515-5520; Yu et al. (2002) Proc Natl Acad Sci USA 99:6047-6052;
(2) expressing short hairpin RNAs from plasmid systems using RNA polymerase III (pol III) promoters. See, for example, Kawasaki et al., supra; Miyagishi et al., supra; Lee et al., supra; Brummelkamp et al., supra; McManus et al., supra), Paddison et al., supra (both); Paul et al., supra, Sui et al., supra; and Yu et al., supra; and/or
(3) expressing short RNA from tandem promoters. See, for example, Miyagishi et al., supra; Lee et al., supra).

When synthesized in vitro, a typical EM scale RNA synthesis provides about 1 mg of siRNA, which is sufficient for about 1000 transfection experiments using a 24-well tissue culture plate format. In general, to inhibit Pten expression in cells in culture, one or more siRNAs can be added to cells in culture media, typically at about 1 ng/ml to about 10 μg siRNA/ml.

For reviews and more general description of inhibitory RNAs, see Lau et al. (2003 Aug) Sci Amer pp 34-41; McManus et al. (2002) Nature Rev Genetics 3, 737-747; and Dykxhoorn et al. (2003) Nature Rev Mol Cell Bio 4: 457-467. For further guidance regarding methods of designing and preparing siRNAs, testing them for efficacy, and using them in methods of RNA interference (both in vitro and in vivo), see, e.g., Allshire (2002) Science 297:1818-1819; Volpe et al. (2002) Science 297:1833-1837; Jenuwein (2002) Science 297:2215-2218; Hall et al. (2002) Science 297 2232-2237; Hutvagner et al. (2002) Science 297:2056-60; McManus et al. (2002) RNA 8:842-850; Reinhart et al. (2002) Genes Dev. 16:1616-1626; Reinhart et al. (2002) Science 297:1831; Fire et al. (1998) Nature 391:806-811:Moss (2001) Curr Biol 11:R772-5:Brummelkamp et al. (2002) Science 296:550-553; Bass (2001) Nature 411 428-429; and Elbashir et al. (2001) Nature 411:494-498; U.S. Pat. No. 6,506,559; Published US Pat App. 20030206887; and PCT applications WO99/07409, WO99/32619, WO 00/01846, WO 00/44914, WO00/44895, WO01/29058, WO01/36646, WO01/75164, WO01/92513, WO 01/29058, WO01/89304, WO01/90401, WO02/16620, and WO02/29858.

Ribozymes and siNAs could take any of the forms, including modified versions, described for antisense nucleic acid molecules; and they can be introduced into cells as oligonucleotides (single or double stranded), or in an expression vector.

In a preferred embodiment, an antisense nucleic acid, siNA (e.g., siRNA) or ribozyme comprises a single stranded polynucleotide comprising a sequence that is at least about 90% (e.g., at least about 93%, 95%, 97%, 98% or 99%) identical to a segment of SEQ ID NO: 1, or to a complement of a segment of SEQ ID NO: 1. As used herein, a DNA and an RNA encoded by it are said to contain the same “sequence,” taking into account that the thymine bases in DNA are replaced by uracil bases in RNA.

Active variants (e.g., length variants, including fragments; and sequence variants) of the nucleic acid-based inhibitors discussed herein are included. An “active” variant is one that retains an activity of the inhibitor from which it is derived (preferably the ability to inhibit expression). It is routine to test a variant to determine for its activity using conventional procedures.

As for length variants, an antisense nucleic acid or siRNA may be of any length that is effective for inhibition of a gene of interest. Typically, an antisense nucleic acid is between about 6 and about 50 nucleotides (e.g., at least about 12, 15, 20, 25, 30, 35, 40, 45 or 50 nt), and may be as long as about 100 to about 200 nucleotides or more. Antisense nucleic acids having about the same length as the gene or coding sequence to be inhibited may be used. When referring to length, the terms bases and base pairs (bp) are used interchangeably, and will be understood to correspond to single stranded (ss) and double stranded (ds) nucleic acids. The length of an effective siNA is generally between about 15 bp and about 29 bp in length, preferably between about 19 and about 29 bp (e.g., about 15, 17, 19, 21, 23, 25, 27 or 29 bp), with shorter and longer sequences being acceptable. Generally, siNAs are shorter than about 30 bases to prevent eliciting interferon effects. An active variant of an siRNA can lack base pairs from either, or both, of ends of the dsRNA; or can comprise additional base pairs at either, or both, ends of the ds RNA, provided that the total of length of the siRNA is between about 19 and about 29 bp, inclusive.

As for sequence variants, it is generally preferred that an inhibitory nucleic acid, whether an antisense molecule, a ribozyme (the recognition sequences), or an siNA, comprise a strand that is complementary (100% identical in sequence) to a sequence of a gene that it is designed to inhibit. However, 100% sequence identity is not required to practice the present invention. Thus, the invention has the advantage of being able to tolerate naturally occurring sequence variations, for example, in human PTEN, that might be expected due to genetic mutation, polymorphism, or evolutionary divergence. Alternatively, the variant sequences may be artificially generated. Nucleic acid sequences with small insertions, deletions, or single point mutations relative to the target sequence can be effective inhibitors.

The degree of sequence identity may be optimized by sequence comparison and alignment algorithms well-known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). At least about 90% sequence identity is preferred (e.g., at least about 92%, 95%, 98% or 99%), or even 100% sequence identity, between the inhibitory nucleic acid and the targeted sequence of targeted gene.

Alternatively, an active variant of an inhibitory nucleic acid of the invention is one that hybridizes to the sequence it is intended to inhibit under conditions of high stringency. For example, the duplex region of an siRNA may be defined functionally as a nucleotide sequence that is capable of hybridizing with a portion of the target gene transcript under high stringency conditions (e.g., 400 mM NaCl, 40 mM PIPES pH 6.4, 1 mM EDTA, 50° C. or 70° C., hybridization for 12-16 hours), followed generally by washing. Methods for administering a pharmaceutical composition or compound to the patient are known in the art. For example, shRNA can be delivered to a mammalian cell by a vector, such as an adenovirus or retrovirus, in a target-specific manner. (Mize-Omata, S., Yuichi, O., Shigeru, I., Mize, N., Doi, T. S., Transient strong reduction of PTEN expression by specific RNAi induces loss of adhesion of the cells. Biochem Biophys Res Commun. 2005 Mar 25; 328(4):1034-42; see also Edwin, F., Singh, R., Endersby, R., Baker, S., Patel, T., The tumor suppressor PTEN is necessary for human sprouty 2-mediated inhibition of cell proliferation. J. Biol. Chem., Vol. 281, Issue 8, 4816-4822, Feb. 24, 2006; Lee, S., Choi, E., Jin, C., Kim, D., Activation of PI3K/Akt pathway by PTEN reduction and PIK3CA mRNA amplification contributes to cisplatin resistance in an ovarian cancer cell line. Gynecol Oncol. 2005 Apr; 97(1):26-34; Zhang, C., Comai, L., Johnson, D., PTEN represses RNA polymerase I transcription by disrupting the SL1 complex. Molecular and Cellular Biology, Sug. 2005, 6899-6911.) Those sequences that show RNAi activity could be recloned into a replication-defective human adenovirus serotype 5 (Ad5). One reason for selection of this viral vector would be the high titer obtainable (in the range of 10¹⁰) and therefore the high multiplicities-of infection that could be attained. Another advantage of this virus would be the high susceptibility and infectivity and the host range (with respect to cell types).

Retroviral vectors used to express shRNA could be put under the control of an RNA polymerase III (pol III) promoter, such as U6 or H1 for the purpose of inhibiting gene expression in a sequence specific manner. Several replication-competent retroviral vectors have been developed for this general purpose. (Bromberg-White, J. L., Webb, C. P., Patacsil, V. S., Miranti, C. K., Williams, B. O., and Holman, S. L., Delivery of short hairpin RNA sequences by using a replication-competent avian retroviral vector. 2004 Journal of Virology, May:4914-4916.)

Thus, the inventors have discovered that the inhibition or inactivation of PTEN in osteoblasts results in enhanced bone formation. In that regard, the present invention includes methods for screening for osteogenic compounds, including selecting a compound that may be a PTEN antagonist, performing a bone formation assay on that compound, and concluding from the assay whether the compound is osteogenic. As exemplified by the Examples below, either in vivo or in vitro bone formation assays can be used to determine whether the selected compound causes bone formation or bone repair in mammals (i.e., humans or animals). The selected compound is osteogenic if the results of the bone formation assay indicate a significant statistical difference in bone formation as compared to a control. Because both osteoblasts and osteoblast precursors demonstrate PTEN expression, either cell type may be used in the screening method.

The present invention also includes a pharmaceutical composition for enhancing bone formation, as well as a method for treating a condition associated with bone loss by administering the pharmaceutical composition. In a preferred embodiment, the pharmaceutical composition reduces the phosphatase activity of PTEN in osteoblast or osteoblast precursors. Such pharmaceutical compositions can be provided to a patient in need of bone formation or to a patient having a bone disease or condition, and can be formulated for human and/or animal use. Various techniques for formulating such compositions are known. Remington's Pharmaceutical Sciences. Frequency, dosage, duration, mode, and route of administration may be varied depending on the patient and condition.

The preferred animal subject of the present invention is a mammal. The invention is particularly useful in the treatment of human subjects. By the term “treating” or “treatment” is intended the administering to a subject of an effective dose of a pharmaceutical composition comprising a PTEN siRNA or other PTEN-specific siNA, preferably in the form of a viral vector that comprises (a) an expression construct of the siRNA operatively linked to a promoter, and (b) a pharmaceutically acceptable excipient or carrier. For administration of the composition to animal or human patients, the dosage amount may vary between 0.01 through 100 mg/kg and may be administered once or repeatedly. With respect to oral administration, bioavailability must be considered. Further, the pharmaceutical compounds of the present invention can be administered in the form of a syrup, capsule, tablet, or caplet. It is anticipated that the pharmaceutical composition may be used with one or more other compounds in combination.

In another preferred method of the invention, particularly with respect to bone repair, the pharmaceutical compound may be administered topically or locally where the bone formation is desired. Such method may be by injection or by transdermal administration. For injection, the pharmaceutical compositions can be formulated as liquid solutions or suspensions with excipients such as water, saline, dextrose, glycerol, or similar excipients.

Having now generally described the invention, the same will be more readily understood through reference to the following examples, which are provided by way of illustration, and are not intended to be limiting of the present invention, unless specified.

### EXAMPLES

### Example 1

### Generation of Osteoblast-Specific, Pten-Deficient Mice

Mice lacking Pten in osteoblasts were generated by crossing OC-cre mice (Zhang M, Xuan S, Bouxsein M L, von Stechow D, Akeno N, Faugere M C, Malluche H, Zhao G, Rosen C J, Efstratiadis A, Clemens T L, 2002. Osteoblast-specific knockout of the insulin-like growth factor (IGF) receptor gene reveals an essential role of IGF signaling in bone matrix mineralization 10. J Biol Chem 277:44005-44012; Holmen S L, Zylstra C R, Mukheijee A, Sigler R E, Faugere M C, Bouxsein M L, Deng L, Clemens T L, Williams B O, 2005. Essential role of beta-catenin in postnatal bone acquisition. J Biol Chem 280:21162-21168) with homozygous conditional mutants carrying modified PTEN alleles (with loxP sites flanking exon 3) (Suzuki A, Yamaguchi M T, Ohteki T, Sasaki T, Kaisho T, Kimura Y, Yoshida R, Wakeham A, Higuchi T, Fukumotot M, Tsubata T, Ohashi P S, Koyasu S, Penninger J M, Nakano T, Make T W, 2001. T cell-specific loss of Pten leads to defects in central and peripheral tolerance. Immunity 14:523-534), to generate OC-cre/Pten^{flox/flox} progeny (FIG. 1)

This system is based on the P1 bacteriophage to specifically inactivate the Pten gene in osteoblasts. In this system, the P1 bacteriophage cyclization recombination (cre) recombinase mediates recombination between two loxP sites, eliminating the DNA between the two sites (Le, Y. And Saver, B., (2001) Mol Biotechnol, 17:269-75). Two loxP sites were placed into the mouse genome using ES-cell based genetic modification approaches in such a way that they did not interfere with the normal function of the gene of interest. However, because they are designed to flank part of the sequence required for a gene to function, when the loxP-modified cells are exposed to cre recombinase, part of the gene will be eliminated, causing loss of gene function. Genes designed in such a manner are said to be “flanked by loxP sites” or “floxed.”This system also provided the additional advantage of allowing the inventors to look at the effects of isolating the effects of loss of Pten activity exclusively to cells of the osteoblast lineage, allowing them to ascribe phenotypes observed to alterations in this cell type. More specifically, the inventors crossed mice carrying a floxed allele of Pten (Suzuki, A., Yamaguchi, M. T., Ohteki, T., Sasaki, T., Kaisho, T., Kimura, Y., Yoshida, R., Wakeham, A., Higuchi, T., Fukumoto, M., Tsubata, T., Ohashi, P. S., Koyasu, S., Penninger, J. M., Nakano, T. & Mak, T. W. (2001) Immunity 14, 523-34) with a mouse strain (Osteocalcin-cre or OC-cre) that expresses cre recombinase in a osteoblast-specific manner (Zhang, M., Xuan, S., Bouxsein, M. L., von Stechow, D., Akeno, N., Faugere, M. C., Malluche, H., Zhao, G., Rosen, C. J., Efstratiadis, A. & Clemens, T. L. (2002) J Biol Chem 277, 44005-12). This strain is very efficient in inducing recombination of floxed genes specifically in osteoblasts.

FIG. 1 shows the strategy for conditional disruption of Pten. The specificity and efficiency of recombination at several loci was reported previously using these animals (Zhang M, Xuan S, Bouxsein M L, von Stechow D, Akeno N, Faugere M C, Malluche H, Zhao G, Rosen C J, Efstratiadis A, Clemens T L, 2002. Osteoblast-specific knockout of the insulin-like growth factor (IGF) receptor gene reveals an essential role of IGF signaling in bone matrix mineralization 10. J Biol Chem 277:44005-44012). All experiments performed were in compliance with the guiding principles of the “Care and Use of Animals” available at www.nap.edu/books/0309053773/html and approved prior to use by the UAB Institutional Animal Care and Use Committee.

### Example 2

### Genotype Analysis

DNA was prepared from tail biopsies using an AutoGenprep 960 automated DNA isolation system. PCR-based strategies were then used to genotype these mice. The Cre transgene was detected by PCR (1 min at 94° C., 1 min at 53° C., and 1 min at 72° C., for 30 cycles) using the primers 5′-CAAATAGCCCTGGCAGATTC-3′ (forward) and 5′-TGATACAAGGGACATCTTCC-3′ (reverse) to generate a 260-bp product corresponding to a portion of the OC promoter and the rabbit β-globin intron. The status of the Pten locus was determined by PCR with the primers 5′-GTCACCAGGATGCTTCTGAC-3′ (forward) and 5′-GAAACGGCCTTAACGACGTAG-3′ (reverse).

### Example 3

### Analysis of Bone Densitometry Using Dual Energy X-ray Absorptiometry (DEXA)

The inventors used a GE Lunar PIXImus II mouse bone densitometer. Bone mineral density was measured at the femur in control (FIG. 2, left bars for each age) and ΔPten mice (FIG. 2, right bars for each age) mice at 3-16 months of age. Data were acquired from isolated specimens that included the lumbar vertebrae, the pelvic girdle and both hind legs. The resolution of the PIXImus is 0.18×0.18 mm pixels with a usable scanning area of 80×65 mm, allowing for measurement of single whole mice and collections of isolated specimens. Calibrations were performed with a phantom using known values, and quality assurance measurements were performed daily with this same phantom. The precision for BMD is less than 1% for whole body and approximately 1.5% for specialized regions. Mice were placed under isoflourane anesthesia during the DEXA process, which took approximately three minutes. Subcranial bone was assessed in this procedure. The process allowed for quantitative measurement of total body bone mineral density.

### Example 4

### Microcomputed Tomography (μACT)

High resolution images of the femur were acquired using a desktop microtomographic imaging system (MicroCT40; Scanco Medical AG, Basserdorf, Switzerland). The femur was scanned at 45 keV with an isotropic voxel size of 6 μm, and the resulting two-dimensional cross-sectional images are shown in gray scale. Scanning was started in the mid-epiphysis and extended proximally for ˜3.6 mm (600 C).

### Example 5

### Immunohistochemistry (IHC)

IHC was performed according to dilutions and conditions suggested by the manufacturers: OPG (Santa Cruz or R&D Systems), RANKL (Santa Cruz and R&D Systems), and β-catenin (Becton Dickinson).

### Example 6

### Histomorphometry

Measurements were performed of parameters of bone structure (cortical thickness, cancellous bone volume/tissue volume, and trabecular thickness) and bone microarchitecture (bone and marrow star volume, strut analysis, and number of trabecules) using the fully automatic Osteoplan III system, (Kontron, Munich, Germany). Static and dynamic parameters of bone formation and resorption were analyzed using the semiautomatic Osteoplan II system. The following parameters were selected: osteoid volume/bone volume, osteoid surface/bone surface, osteoid thickness, number of osteoblasts/bone perimeter, number of osteocytic lacunae (filled and empty)/trabecular bone area, erosion surface/bone surface, number of osteoclasts/bone perimeter, mineral apposition rate, mineralizing surface/bone surface, bone formation rate/bone surface, bone formation rate/osteoblast, and mineralization lag time. All parameters were calculated according to the histomorphometry nomenclature committee of the American Society of Bone and Mineral Research.

### Example 7

### Dynamic Histomorphometry/Calcein Labeling

To address the in vivo growth rates of bone in the mutants, calcein labeling was performed (FIG. 4A). A calcein solution in PBS (0.8 mg/ml) was prepared and injected into mice at a dose of 25 mg/kg. A second injection at a later timepoint was done and animals were sacrificed shortly thereafter. Bone samples were prepared and the distance between the calcein incorporation as judged by fluorescence microscopy was calculated. This was used to calculate the bone formation rate (BFR). Dynamic indices of bone formation were quantitated in epiphyseal trabeculae of the femur of 6 week old male. The timing between the two injections depended on the age of the mouse.

### Example 8

### Bone Histology

Demineralized Bone Histology—Tissue samples were fixed in formalin overnight, decalcified in Immunocal decalcifying agent (Decal, Baltimore, Md.) overnight, and then dehydrated through a graded alcohol series in a Ventana Renaissance processor (Ventana Medical Systems, Tucson, Ariz.). Tissues were paraffin-embedded, and 5-μm sections were adhered to glass slides. Slides were de-paraffinized and stained with hematoxylon and eosin or left unstained for immunohistochemistry.

Mineralized Bone Histology—Femurs were fixed in ethanol at room temperature, dehydrated, and embedded in methylmethacrylate. 3-μm sections were cut with a Microm microtome and stained with modified Mason-Goldner trichrome stain. The number of osteoblasts and osteoclasts per bone perimeter were measured at standardized sites under the growth plate using a semiautomatic method (Osteoplan II; Kontron, Munich, Germany) at a magnification of ×200. These parameters comply with guidelines of the nomenclature committee of the American Society of Bone and Mineral Research (Parfitt A M, Drezner M K, Glorieux F H, Kanis J A, Malluche H, Meunier P J, Ott S M, Recker R R., Bone histomorphometry: standardization of nomenclature, symbols, and units. Report of the ASBMR Histomorphometry Nomenclature Committee, J Bone Miner Res. 1987 2(6):595-610.).

### Example 9

### Osteoblast Isolation and Culture

Osteoblasts were isolated from calvaria of newborn mice by serial digestion in a minimal essential medium (Mediatech, Herndon, Va.) containing 10% bovine serum albumin, 25 mM HEPES, pH 7.4, 0.2 mg/ml collagenase type I (Worthington, Lakewood, N.J.), 0.7 mg/ml collagenase type 2 (Worthington), and 1 mM CaCl₂. Calvaria were digested for 15 min at 37° C. with constant agitation. The digestion solution was collected, washed with fresh medium, and digested an additional five times. Digestions 3-6 (containing the osteoblasts) were centrifuged, washed with [symbol] -minimal essential medium containing 10% fetal bovine serum, 1% pen/strep, and plated overnight at 37° C. The next day, the cells were trypsinized and 1.1×10⁵ cells were plated on 6-cm dishes. The medium was supplemented with 5 mM β-glycerophosphate (Sigma) and 100 μg/ml ascorbic acid (Sigma) (mineralization medium), which was replaced every other day.

### Example 10

### Measurement of Osteoblast Proliferation

Mitotic index was measured via bromodeoxyuridine (BrdU) labeling (FIG. 7A). BrdU (100 mg/kg) was injected intraperitoneally 96 hours prior to sacrifice. Simultaneous examination of actively dividing cells in bone marrow served as internal controls. Tissues were harvested, decalcified in EDTA, and embedded in paraffin. Peroxidase immunohistochemistry with monoclonal anti-BrdU (Becton-Dickinson, 1:100) was used to identify proliferating cells by nuclear labeling and compared to total cell number using a hematoxylin and eosin counterstain. The osteoblast nuclear labeling index (# labeled nuclei/total nuclei) was compared after appropriate coding of samples to prevent observer bias.

### Example 11

### Purification of mRNA from Calvarial Osteoblasts

RNA isolation was performed using TRIzol (See TRIzol protocol at http://www.invitrogen.com/content/sfs/manuals/15596026.pdf). Total RNA pellets were then suspended in 800 ul nuclease free water and then precipitated in 2.5 M LiCl solution. The samples were then incubated overnight at −20° C. and washed twice with 75% ethanol, 25% Dep-C treated water. Purified RNA was suspended in 250 μl nuclease free water following careful removal of residual ethanol by pipetting, and finally concentrated to 5.6 μg/μl by sodium acetate (Ambion) precipitation and stored at −80 degrees.

### Example 12

### Infection of Osteoblasts with Adenovirus

Adenoviral constructs were introduced into osteoblasts 24 hours after initial plating. Constructs were prepared in 293 cells using the adeno-bacterial vector plasmid pJM17, containing most of the adenoviral genome in a circular form with a deleted E1A and E1B region. A construct with the gene of interest (Cre recombinase) driven by the CMV promoter was then inserted into adeno virus transfer vector pMV60 by restriction enzyme digestion and ligation. This plasmid was then co-transfected into the 293 cell line along with plasmid pJM17 to generate recombinant adenovirus expressing Cre. Plaques were isolated, expanded and analyzed by restriction enzyme digestion and/or PCR. The recombinant adenovirus was propagated in 293 cells and used in primary cell culture systems. Cells were infected with recombinant adeno virus (1 pfu/cell) in DMEM with 2% FBS for 30-60 minutes after which the cells were allowed to grow in DMEM with 10% FBS for 2-3 days or until all the cells show the cytopathic effect. To obtain the virus, the infected cells were harvested, centrifuged, and the pellet dissolved in DMEM. The cells were then subjected to three cycles of freezing and thawing to release the virus. The virus could then be titrated by plaque assay and used in the desired concentration directly on primary osteoblasts.

### Example 13

Von Kossa Staining—Cultures were maintained in differentiation medium for the days indicated and fixed in 10% neutral buffered formalin. The cells were washed with water, dehydrated, and allowed to air dry. Silver nitrate (2%) was added to the cells for 20 min. The cells were washed with water and then incubated with 5% sodium carbonate for 3 min.

### Example 14

### Protein Extracting and Immunoblotting

Proteins were extracted from cultured osteoblasts by using M-PER (Pierce, Rockford, Ill.) with protease inhibitors (Pierce). Cell lysates and immunoprecipitates were separated on 10% SDS polyacrylamide gels and transferred to nitrocellulose membranes. The membranes were probed using an anti-β-catenin monoclonal antibody (BD Transduction Laboratories). The membranes were then incubated with horseradish peroxidase-linked secondary anti-mouse antibodies and bound antibodies were visualized using the Supersignal West Femto Maximum Sensitivity Substrate (Pierce).

### Example 15

### Osteoblast Apoptosis

Osteoblasts were plated on 100-mm plates (1.0×10⁶ per plate) and cultured in αMEM with 1% FBS for 48 h and serum-free ΔMEM for 12 h before harvesting cells for annexin V-PE (BD PharMingen) staining. After staining with annexin V-PE, cells were analyzed immediately by FACSCalibur (Becton Dickinson), and 20,000 events were collected.

### Example 16

### Alkaline Phosphatase Staining

Alkaline phosphatase activity was measured in cell layers as previously described by using a p-nitrophenyl phosphate substrate and an incubation temperature of 37° C. (41). Two days after adenovirus treatment, osteoblasts were replated in six-well plates with 1.0×10⁵ cells per well. Cells were cultured in αMEM with 10% FBS (GIBCO, Carlsbad, Calif.) and supplemented with L-ascorbic acid (50 μg/ml; Sigma, St. Louis, Mo.) and β-glycerol phosphate (10 mM; Sigma) for 21 days. Media were changed every other day.

### Example 17

### Real-Time PCR

Total RNA was extracted from cells by using the TRIzol (Life Technologies, Carlsbad, Calif.) extraction protocol. First-strand cDNA was synthesized by using the Invitrogen kit. Real-time PCR was performed at 57° C. for 30 cycles in the Opticon Continuous Fluorescent Detector by using IQ SYBR Green supermix (Bio-Rad, Hercules, Calif.). Samples were run in triplicate and the results were normalized to β-actin expression. Primer sequences used were as follows: Pten, F5′-CATAACCCACCACAGCTAG-3′ and R5′-GCAGACCACAAACTGAGG-3′; Runx-2, F5′-CCAAATTTGCCTAACCAGAATG-3′ and R5′-GAGGCTGTGGTTTCAAAGCAC-3′; NaPi-3, F5′-CACCCATATGGCTTCTGCTT-3′ and R5′-CAGGAATTCATAGCCCAGGA-3′; Glut-1, F5′-CAGTTTCGAGAAGAACATGAG-3′ and R5′-GC-GGAATTCAATGCTGATGAT-3′; PGK, F5′-GGAAGCG-GGTCGTGATGA-3′ and R5′-GCCTTGATCCTTTGGTTGTTTT-3′; β-actin, F5′-CTGAACCCTAAGGCCAACCGTG-3′ and R5′-GGCATACAGGGACAGCACAGCC-3′.

### Example 18

### Statistics

For in vitro studies, standard descriptive statistics were computed for all outcomes. To compare two groups of data, the Student's t-test was used. For multiple comparisons between more than two groups, data was analyzed by one-way analysis of variance (ANOVA), followed by Dunnett's test. Significant differences were considered when a p<0.05 was obtained. For in vivo studies, the histomorphometric data displayed the most variability. Thus, the inventors performed power calculations to estimate the numbers of mice required for detection of significant changes. In a cross-sectional design, the greatest variance observed among the selected histomorphometric parameters of bone is 14.63 for number of osteoblasts/bone perimeter. Based on previous studies, the average expected detectable difference was 10.8%. With an 80% power, at 0.05 level of significance, the number of animals per group required for the study was 10. Group descriptive statistics were performed on all variables. Normality of distribution were assessed by the Lilliefors test, and homogeneity of variance tested with the Levene test. Adequate transformations of the data were done when results did not meet the characteristics assumed for analysis of variance (SPSS; Chicago, Ill.). Comparability of results obtained in cross-sectional design between groups was done using analysis of variance with the Scheffe a posteriori test.

### Example 19

### Allele-Specific PCR Detection of the ΔPten Allele in Mutant and Control Mice

DNA was isolated from the various tissues noted by Proteinase K digestion of these tissues followed by purification of DNA using isopropanol precipitation and subsequent resuspension of the DNA in TE buffer. The samples were then assessed by a specific PCR reaction which gives a band when cre-mediated recombination has occurred. A band of approximately 500 base pairs indicates that cre-mediated recombination has occurred in that tissue, while a band of approximately 1300 base pairs indicates the presence of DNA that has not recombined. The only recombination detected was in tissues which contain osteoblasts.

### Example 20

### Establishment of Adeno-Cre Viral Titer for Pten Gene Deletion

Primary Pten^{flox/flox} osteoblasts were infected with the indicated PFU (plaque forming units) of Adeno-Cre or Adeno-GFP virus. Total RNA was extracted from cells by using the TRIzol (Life Technologies, Carlsbad, Calif.) extraction protocol. First-strand cDNA was synthesized by using the Invitrogen kit. Real-time PCR was performed at 57° C. for 30 cycles in the Opticon Continuous Fluorescent Detector by using IQ SYBR Green supermix (Bio-Rad, Hercules, Calif.). Samples were run in triplicate and the results were normalized to beta<https://email.vai.org/math/beta.gif>-actin expression. RNA was isolated from these cells 48 hours after infection and then real-time PCR was used to measure relative Pten gene expression.

### Example 21

### Cell Size Measurement

Control and Pten-deficient osteoblasts were analyzed by flow cytometry with forward and side scatter (FIGS. 7E and 7F). A small laser beam of very bright light hits the cells as they pass through the flow chamber. The manner in which the light bounces off each cell provides information about the cell's physical characteristics. Light bounced off at small angles is called forward scatter. Light bounced off in other directions is called side scatter. FIG. 7F shows the data of FIG. 7E in graphical format.

### Example 22

### Measurement of Pten Deletion on Osteoblast Differentiation and Alkaline Phosphatase Expression

Total RNA was extracted from cells by using the TRIzol (Life Technologies, Carlsbad, Calif.) extraction protocol. First-strand cDNA was synthesized by using the Invitrogen kit. Real-time PCR was performed at 57° C. for 30 cycles in the Opticon Continuous Fluorescent Detector by using IQ SYBR Green supermix (Bio-Rad, Hercules, Calif.). Samples were run in triplicate and the results were normalized to beta<https://email.vai.org/math/beta.gif>-actin expression. Primer sequences used were as follows: Pten, F5′-CATAACCCACCACAGCTAG-3′ and R5′-GCAGACCACAAACTGAGG-3′; Runx-2, F5′-CCAAATTTGCCTAACCAGAATG-3′ and R5′-GAGGCTGTGGTTTCAAAGCAC-3′; NaPi-3, F5′-CACCCATATGGCTTCTGCTT-3′ and R5′-CAGGAATTCATAGCCCAGGA-3′; Glut-1, F5′-CAGTTTCGAGAAGAACATGAG-3′ and R5′-GC-GGAATTCAATGCTGATGAT-3′; PGK, F5′-GGAAGCG-GGTCGTGATGA-3′ and R5′-GCCTTGATCCTTTGGTTGTTTT-3′; beta<https://email.vai.org/math/beta,gif>-actin, F5′-CTGAACCCTAAGGCCAACCGTG-3′ and R5′-GGCATACAGGGACAGCACAGCC-3′.

What is claimed is:

1. A method of screening for osteogenic compounds comprising the following steps:
selecting a compound that may be a PTEN antagonist;
performing a bone formation assay for the compound; and
concluding from the assay whether the compound is osteogenic.

2. The method of claim 1, wherein the selected compound antagonizes PTEN in an osteoblast or osteoblast precursor.

3. The method of claim 1, wherein the bone formation assay measures bone mass.

4. The method of claim 1, wherein the bone formation assay measures bone volume.

5. The method of claim 1, wherein the bone formation assay measures osteoblast number.

6. The method of claim 1, wherein the bone formation assay measures osteoblast survival.

7. The method of claim 1, wherein the conclusion from the bone formation assay is that the selected compound is osteogenic.

8. The method of claim 7, wherein the selected compound inactivates PTEN.

9. The method of claim 7, wherein the selected compound is an siNA for PTEN.

10. The method of claim 7, wherein the compound interacts with the phosphatase domain of PTEN.

11. The method of claim 9 wherein the siNA is an shRNA.

12. A pharmaceutical composition for enhancing bone formation comprising a compound that lowers the phosphatase activity of PTEN in osteoblasts or osteoblast precursors.

13. The pharmaceutical composition of claim 12, wherein the compound inactivates PTEN in osteoblasts or osteoblast precursors.

14. The pharmaceutical composition of claim 12, wherein the compound is an siNA for PTEN.

15. The pharmaceutical composition of claim 12, wherein the compound interacts with the phosphatase domain of PTEN.

16. The pharmaceutical composition of claim 14, wherein the siNA is an shRNA.

17. A method of enhancing bone formation in a mammal comprising administering to the mammal an effective amount of an osteoblast-specific PTEN antagonist.

18. The method of claim 17, wherein the osteoblast-specific PTEN antagonist inactivates PTEN in osteoblasts or osteoblast precursors in the mammal.

19. The method of claim 17, wherein the osteoblast-specific PTEN antagonist is an siNA for PTEN.

20. The method of claim 17, wherein the antagonist interacts with the phosphatase domain of PTEN.

21. The method of claim 19, wherein the siNA is an shRNA.

22. A method of treating a condition associated with loss of bone mass comprising administering a pharmaceutical compound that reduces the phosphatase activity of PTEN in osteoblasts or osteoblast precursors.

23. The method of claim 22, wherein the compound is administered in an amount effective to enhance bone formation.

24. The method of claim 23, wherein the compound is administered in an amount effective to increase bone mass.

25. The method of claim 23, wherein the compound is administered in an amount effective to increase bone volume.

26. The method of claim 23, wherein the compound is administered in an amount effective to increase osteoblast number.

27. The method of claim 23, wherein the compound is administered in an amount effective to increase osteoblast survival.

28. The method of claim 22, wherein the compound is an siNA for PTEN.

29. The method of claim 22, wherein the compound interacts with the phosphatase domain of PTEN.

30. The method of claim 28, wherein the siNA is an shRNA.
